(19) United States
(12) Patent Application Publication
(10) Pub. No.: US 2024/0108794 A1
(43) Pub. Date: Apr. 4, 2024

(54) LOW FLOW SWITCH FOR MEDICAL ASPIRATION

(71) Applicant: Medtronic, Inc., Minneapolis, MN (US)

(72) Inventors: Rónán Wood, Galway (IE); Erico Cruz, Galway (IE); Julio Jenaro, Galway (IE)

(21) Appl. No.: 17/937,245

(22) Filed: Sep. 30, 2022

(51) Int. Cl. A61M 1/00

(52) U.S. Cl. CPC A61M 1/743; A61M 1/80

(57) ABSTRACT

An example medical device for aspirating material from a patient includes a flow switch including an anvil, an actuator, and a surface feature on at least one of the anvil and actuator. The flow switch is configured to move the actuator away from the anvil create a flow path for the aspiration of the material and to move the actuator toward the anvil to reduce the flow path by creating at least one channel defined by the surface feature.

### TECHNICAL FIELD

This disclosure relates to medical aspiration.

### BACKGROUND

In some cases, medical aspiration can be used to remove material from a patient. For example, medical aspiration can be used to remove a thrombus, such as a clot or other occlusion, from a blood vessel of a patient.

### SUMMARY

This disclosure describes example devices and systems configured to reduce withdrawal of body fluid (e.g., blood) from a patient during a medical aspiration procedure, and related methods. An aspiration catheter can be used to remove a thrombus from a hollow anatomical structure (e.g., a blood vessel) of a patient. For example, a distal opening of the catheter may be positioned in the hollow anatomical structure near a thrombus and an aspiration force can be applied to a lumen of the aspiration catheter in order to draw the thrombus through the catheter lumen and out of the hollow anatomical structure. In some instances, such as with deep vein thrombosis (DVT) and pulmonary embolism (PE) procedures, it may be preferable to use larger aspiration catheters (e.g., 12 French or greater) to remove large clot burdens from vessels. As the aspiration catheter lumen diameter increases, the flow through the aspiration catheter increases, shortening the time in which the maximum blood volume that can be extracted from a patient (approx. 450 milliliters) will be reached more quickly than in a case with a smaller aspiration catheter, and potentially force a premature end to the procedure.

In examples described herein, an aspiration system may be configured to reduce blood loss during a procedure, even with a larger aspiration catheter (e.g., 12 French or greater, or any size catheter) by controlling the flow and/or amount of blood through the aspiration catheter. For example, the aspiration system may monitor blood flow using a sensor and actuate a control valve based on the measured flow. The control valve may allow unrestricted flow to pass when the distal end of the aspiration catheter encounters a blockage (from thrombus or potentially a vessel wall) but will regulate (e.g., reduce) the flow of blood if the aspiration catheter is in patent blood flow in a vessel. In some examples, the aspiration system may allow a physician to “search” for thrombus in the vessel using fluoroscopy guidance, while substantially only drawing high blood flow when the catheter tip is occluded.

In examples described herein, an aspiration system includes an aspiration catheter fluidically coupled to a flow switch (also referred to herein as a valve) configured to regulate the flow of a fluid (e.g., blood) through a lumen of the catheter during a medical procedure to allow a relatively low flow of the fluid through the lumen when in a low flow configuration. In the low flow configuration of the flow switch, the flow switch compresses aspiration tubing, which can include the catheter or other tubing in fluid communication with the catheter. The low flow configuration may also be referred to herein as a closed, collapsed, or compressed configuration, albeit in the examples described herein there is still some fluid flow allowed when in such a “closed” configuration.

In some examples, the flow switch comprises an actuator and an anvil configured to form a pinch valve operable to collapse a lumen of a flexible tube (e.g., configured to be in fluid communication with a catheter lumen of the catheter or the catheter itself) when in a low flow configuration. The anvil or the actuator may include a surface feature configured to cause a channel within the lumen of the flexible tube to remain open when compressing and/or collapsing the lumen of the flexible tube, e.g., in the low flow configuration.

The devices, systems, and techniques of this disclosure may provide one or more advantages and benefits. For instance, allowing some flow of fluid through the catheter lumen when the flow switch is in the low flow configuration may reduce a volume of body fluid withdrawn from the body during an aspiration procedure while reducing blockages in the system compared to when the flow of fluid through the catheter lumen is completely stopped. Enabling some fluid flow through the catheter lumen may help reduce clot formation within the aspiration catheter and/or tubing of the system, as well as keep an increased pressure within the discharge reservoir which may reduce boiling and/or foaming of the fluid within the discharge reservoir, e.g., to prevent the fluid from reaching the top and/or lid of the discharge reservoir container.

Additionally, a flow switch including only two positions, open and closed, as described in some examples herein, simplifies the flow switch and can provide better control than a flow switch that can only enable low flow through a catheter lumen by actuating to an intermediate state between a fully open state and a fully closed state, e.g., to partially close the lumen of the flexible tubing. For example, the tolerances required for such a flow switch to partially close the lumen of a flexible tube may be significantly tighter (e.g., increased tolerancing) in order to achieve an acceptable low flow repeatability. Additionally, flexible tubing may change over time, e.g., the durometer of the material may change over repeated compressions and releases, or the tubing may be replaced with different tubing (e.g., having a different size, wall thickness, lumen size, material, or replacement tubing of the same type), which may respond to compression and/or release differently, and even with tight tolerancing. As a result of the change in flexible tubing over time and a change in a response of the flexible tubing to a compressive force, the low flow rate of a flow switch configured to partially close the lumen of the flexible tube by actuating to an intermediate state may drift over time. By contrast, and in accordance with the techniques, devices, and systems disclosed herein, in a closed state of an example flow switch described herein, some fluid flow through the catheter lumen is permitted. For example, the flow switch can be configured to fully collapse the lumen of a flexible tube via compressing the flexible tube between an actuator and an anvil, and either or both the actuator and the anvil includes a surface feature configured to cause a channel within the lumen of the flexible tube to remain open even when the flow switch is in the closed (and not an intermediate) state. This can provide improved repeatability and longevity of achieving a particular low flow rate.

In one example, this disclosure describes a medical device for aspirating material from a patient, the device including: a flow switch including: an anvil; an actuator; and a surface feature on at least one of the anvil and actuator, wherein the flow switch is configured to: move the actuator away from the anvil create a flow path for the aspiration of the material; and move the actuator toward the anvil to reduce the flow path by creating at least one channel defined by the surface feature.

In another example, this disclosure describes a medical aspiration system including: a suction source; an elongated body defining a lumen fluidically coupled to the suction source; and a pinch valve configured to actuate between a high flow configuration and a low flow configuration, wherein in the low flow configuration, the pinch valve is configured to compress the elongated body while still enabling fluid flow through the lumen.

In another example, this disclosure describes a method for aspirating material from a patient using a flow switch, an anvil, an actuator, and a surface feature on at least one of the anvil and actuator, the method including: opening the flow switch by moving the actuator away from the anvil to create a flow path for the aspiration of the material; and closing the flow switch by move the actuator toward the anvil to reduce the flow path by creating at least one channel defined by the surface feature.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic diagram illustrating an example aspiration system including a passive flow switch.

FIG. 2A is a schematic side view of an example of an example flow switch in a “high flow” configuration.

FIG. 2B is a schematic side view of an example of the flow switch of FIG. 2A in a “low flow” configuration.

FIG. 3A is a schematic cross-sectional top view of an example of the flow switch of FIG. 2A taken along the line A-A′.

FIG. 3B is an expanded schematic side view of the example actuator and anvil of the flow switch of FIG. 2B in the low flow configuration.

FIG. 4A is a schematic side view of an example flow switch.

FIG. 4B is a schematic front view of an example of the flow switch of FIG. 4A.

FIG. 4C is a schematic side view of an example of the flow switch of FIG. 4A.

FIG. 5 is an expanded schematic side view of another example actuator and anvil of another example flow switch in a low flow configuration.

FIG. 6 is a schematic front view of an example of the flow switch of FIG. 1 in a high flow configuration.

FIG. 7 is an expanded schematic side view of the example actuator and anvil of the flow switch of FIG. 6 in the low flow configuration.

FIG. 8 is an expanded schematic side view of an example flexible tube between the example actuator and anvil of the flow switch of FIG. 2A in the low flow configuration.

### DETAILED DESCRIPTION

This disclosure describes devices and systems configured to regulate a flow of a body fluid, such as blood, from a body of a patient during a medical aspiration procedure, as well as medical aspiration systems (e.g., vascular aspiration systems) including such devices and systems, and corresponding methods. In examples described herein, an aspiration system includes a flow switch (also referred to herein as a “valve”) configured to control a fluid flow rate through a catheter lumen of a catheter by compressing flexible tube. The flexible tube can be the catheter or another elongated body (e.g., aspiration tubing) in fluid communication with the catheter lumen. The flow switch is configured to actuate between an open configuration and a closed configuration (also referred to as open and closed states, respectively), in which the flow switch allows some fluid flow through the catheter lumen. The flow rate through the catheter lumen when the flow switch is in the closed position is greater than zero but less than the flow rate through the catheter lumen when the flow switch is in the open state relative to substantially the same suction force being applied in the open and closed states, e.g., less than or equal to 99% the flow rate through the catheter lumen when the flow switch is in the open state, less than or equal to 50% the flow rate through the catheter lumen when the flow switch is in the open state, less than or equal to 10% the flow rate through the catheter lumen when the flow switch is in the open state, less than or equal to 5% the flow rate through the catheter lumen when the flow switch is in the open state, or less than or equal to 1% the flow rate through the catheter lumen when the flow switch is in the open state.

In some examples, the flow switch comprises a motivator configured to actuate an actuator (e.g., cause the actuator to move) and an anvil configured to form a “pinch” valve operable to collapse a lumen of a flexible tube via compressing the flexible tube and having a surface feature configured to cause a channel within the lumen of the flexible tube to remain open even when the actuator and the anvil cannot be moved closer together to define a further closed state of the flexible tube. In some examples, the motivator may comprise a solenoid, e.g., the actuator includes a solenoid-actuated actuator. In other examples, the actuator may include a pneumatic actuator, a hydraulic actuator, a stepper actuator (e.g., driven by a stepper motor), a servo motor, or any suitable motivator configured to cause the actuator to move. In some examples, the surface feature comprises a recess defined by the surface of one or both of the actuator and anvil. For example, the recess may extend inwardly from the surface of the actuator or anvil, e.g., into the actuator or anvil. In some examples, the surface feature comprises a protrusion extending from a surface of the actuator and/or extending from a surface of the anvil. For example, the protrusion may extend outwardly from the surface of the actuator or anvil. In some examples, the surface feature includes a channel within the anvil, or a plurality of offset anvils, configured to cause the flexible tube to serpentine or “kink” when the actuator is in the low flow (e.g., compressed/collapsed/closed) configuration.

Various aspects of the present disclosure can be useful for aspiration thrombectomy procedures that treat vessel blockages due to thrombus build up in vessel beds including, for example, neurovascular, deep vein thrombosis (DVT), peripheral artery disease, and pulmonary embolism (PE). Some indications, such as DVT and PE, it can be preferable to use larger aspiration catheters (12F and above). The larger catheter sizes can facilitate the removal of large clot burden from vessels. Larger the aspiration catheter internal diameters allow for higher flow rates during aspiration. As a result of the higher flow rates, the more blood volume can be extracted from a patient during a procedure. The recommended limit of the amount of blood extracted (e.g., approx. 450 ml) can be reached more quickly than in a case with a smaller aspiration catheter. This can result in the physician having less time to complete the thrombectomy procedure before reaching such a limit.

Aspects of the present disclosure can be useful in the context of a Blood Loss Mitigation System (BLoMS) that is configured to reduce blood loss during the procedure by controlling and reducing the flow of blood through the aspiration catheter using a switch. For example, the BLoMS can monitor fluid flow using a sensor and actuate a switch based on the measured flow. The BLoMS switch can be opened, to allow unrestricted flow to pass, when the distal end of the aspiration catheter encounters a blockage (such as from thrombus or vessel wall) while closing reducing the flow if the aspiration catheter is in patent blood flow in a vessel, reducing the flow of blood. The operation of the BLoMS will allow a physician to “search” for thrombus in the vessel using fluoroscopy guidance, while only drawing high flow when the catheter tip is occluded.

According to certain aspects of the present disclosure, the BLoMS can be configured to allow for a small amount of fluid or material to flow when the switch is in the closed position. Without being limited by theory, the use of a low flow condition when the BLoMS is actively engaged in the reduction of blood flow can be useful for detecting when the catheter tip engages thrombus and the BLoMS system can be activated. For instance, a flow rate sensor could monitor the flow rate in the low flow rate and detect when the flow stops or significantly decreases, which can indicate blockage of the catheter tip by thrombus.

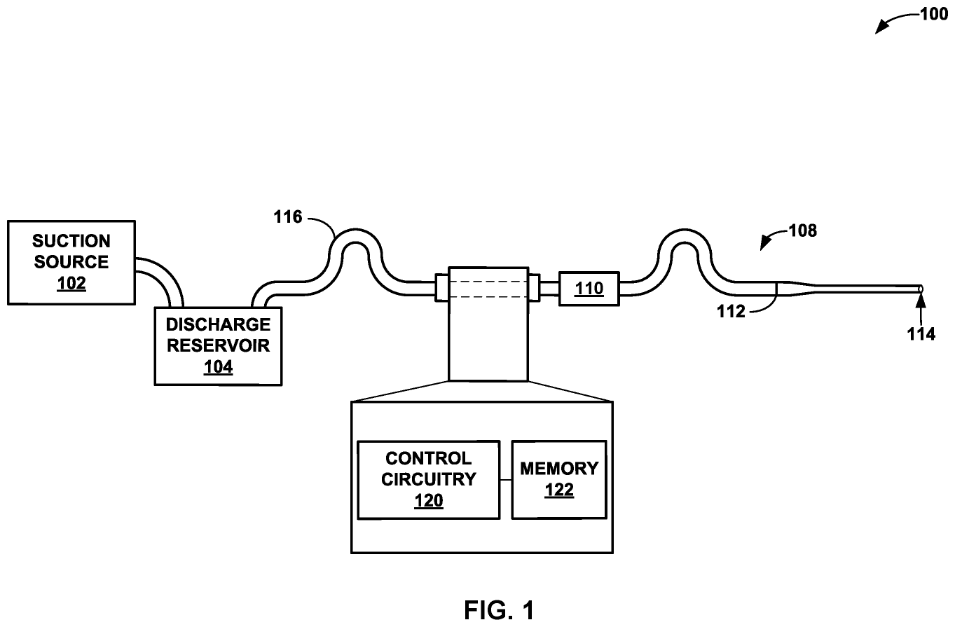

FIG. 1 is a schematic diagram illustrating an example medical aspiration system 100 including a suction source 102, a discharge reservoir 104, a catheter 108 (also referred to herein as “aspiration catheter 108”), and a flow switch 110 (e.g., also referred to herein as “pinch valve 110”). Medical aspiration system 100 may be used to treat a variety of conditions, including thrombosis. Thrombosis occurs when a thrombus (e.g., a blood clot or other material such as plaques or foreign bodies) forms and obstructs vasculature of a patient. For example, medical aspiration system 100 may be used to treat a pulmonary embolism or deep vein thrombosis, which may occur when a thrombus forms in a deep vein of a patient, such as in a leg of the patient.

Medical aspiration system 100 is configured to remove a thrombus from a patient. Medical aspiration system 100 may be configured to remove a thrombus by via catheter 108, e.g., to draw the thrombus from the patient using a suction force applied to catheter 108. Material passing through catheter 108 is deposited into discharge reservoir 104, via a suction force applied by suction source 102 to catheter 108 (e.g., to an inner lumen of catheter 108). Catheter 108 includes an elongated body 112 defining a catheter lumen (not shown in FIG. 1) and terminating in a distal opening 114. To treat a patient with thrombosis, a clinician may position distal opening 114 of catheter 108 in a blood vessel of the patient near the thrombus or other occlusion and apply a suction force (also referred to herein as suction, vacuum force, negative pressure, or aspiration force) to the catheter 108 (e.g., to one or more lumens of the catheter) to engage the thrombus with suction force at distal opening 114 of catheter 108. For example, suction source 102 can be configured to create a negative pressure within the inner lumen of catheter 108 to draw a material from the inside the blood vessel into the catheter lumen via distal opening 114 of catheter 108. The negative pressure within the inner lumen can create a pressure differential between the inner lumen and the environment external to at least a distal portion of catheter 108 that causes the material, e.g., a thrombus, fluid (e.g., blood, saline introduced into the patient as part of the aspiration procedure, or the like), and/or other material, to be introduced from the blood vessel into the catheter lumen via catheter distal opening 114. For example, the fluid may flow from patient vasculature, into the catheter lumen via distal opening 114, and subsequently through aspiration tubing 116 (also referred to herein as “vacuum tube 116”) into discharge reservoir 104.

Once distal opening 114 of aspiration catheter 108 has engaged a thrombus that is within a blood vessel, the clinician may remove aspiration catheter 108 with the thrombus held within opening 114 or attached to the distal tip of elongated body 112, or suction off pieces of the thrombus (or the thrombus as a whole) until the thrombus is removed from the blood vessel of the patient through a lumen of aspiration catheter 108 itself and/or through the lumen of an outer catheter in which aspiration catheter 108 is at least partially positioned. The outer catheter can be, for example, a guide catheter configured to provide additional structural support to the aspiration catheter. In some cases, aspiration of thrombus can be performed concurrently with use of a thrombectomy device, such as a thrombus removal basket, to facilitate removal of thrombus via mechanical thrombectomy as well as via aspiration.

As used herein, “suction force” is intended to include, within its scope, related concepts such as suction pressure, vacuum force, vacuum pressure, negative pressure, and the like. A suction force can be generated by a vacuum, e.g., by creating a partial vacuum within a sealed volume fluidically connected to catheter 108, or by direct displacement of liquid in catheter 108 and/or tubing 116 via (e.g.) a peristaltic pump, or otherwise. Accordingly, suction forces or suction as specified herein can be measured, estimated, computed, etc. without need for direct sensing or measurement of force. A “higher,” “greater,” or “larger” (or “lower,” “lesser,” or “smaller”) suction force described herein may refer to the absolute value of the negative pressure generated by the suction source on a catheter or another component, such as a discharge reservoir 104.

In some examples, suction source 102 can comprise a pump (also referred to herein as “pump 102” or “vacuum source 102”). The suction source 102 can include one or more of a positive displacement pump (e.g., a peristaltic pump, a rotary pump, a reciprocating pump, or a linear pump), a direct-displacement pump (e.g., a peristaltic pump, or a lobe, vane, gear, or piston pump, or other suitable pumps of this type), a direct-acting pump (which acts directly on a liquid to be displaced or a tube containing the liquid), an indirect-acting pump (which acts indirectly on the liquid to be displaced), a centrifugal pump, and the like. An indirect-acting pump can comprise a vacuum pump, which displaces a compressible fluid (e.g., a gas such as air) from the evacuation volume (e.g., discharge reservoir 104, which can comprise a canister), generating suction force on the liquid. Accordingly, the evacuation volume (when present) can be considered part of the suction source. In some examples, suction source 102 includes a motor-driven pump, while in other examples, suction source 102 can include a syringe, and mechanical elements such as linear actuators, stepper motors, etc. As further examples, the suction source 102 could comprise a water aspiration venturi or ejector jet.

Medical aspiration system 100 includes control circuitry 120 configured to control a suction force applied by suction source 102 to catheter 108. For example, control circuitry 120 can be configured to directly control an operation of suction source 102 to vary the suction force applied by suction source 102 to the inner lumen of catheter 108, e.g. by controlling the motor speed, or stroke length, volume or frequency, or other operating parameters, of suction source 102. As another example, control circuitry 120 can be configured to control one or more functions of flow switch 110. Other techniques for modifying a suction force applied by suction source 102 to the inner lumen of catheter 108 can be used in other examples.

Control circuitry 120, as well as other processors, processing circuitry, controllers, control circuitry, and the like, described herein, may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, control circuitry 120 may include multiple components, such as any combination of one or more microprocessors, one or more DSPs, one or more ASICs, or one or more FPGAs, as well as other discrete or integrated logic circuitry, and/or analog circuitry. In some examples, control circuitry 120 may further include, additionally or alternatively to electric-based processors, one or more controls that operate using fluid motion power (e.g., hydraulic power) in combination with or in addition to electricity. For example, control circuitry 120 can include a fluid circuit comprising a fluid circuit comprising a plurality of fluid passages and switches arranged and configured such that, when a fluid (e.g., liquid or gas) flows through the passages and interacts with the switches, the fluid circuit performs the functionality of control circuitry 120 described herein.

Memory 122 may store program instructions, such as software, which may include one or more program modules, which are executable by control circuitry 120. When executed by control circuitry 120, such program instructions may cause control circuitry 120 to provide the functionality ascribed to control circuitry 120 herein. The program instructions may be embodied in software and/or firmware. Memory 122, as well as other memories described herein, may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Although control circuitry 120 and memory 122 are shown in FIG. 1 as being in a common housing, in other examples, control circuitry 120 and/or memory 122 can be physically separate from each other.

With some other aspiration systems, some amount of a body fluid may be incidentally withdrawn during the aspiration procedure. For instance, while approaching and aspirating a thrombus with a distal opening of the catheter, the clinician may incidentally aspirate and remove a volume of the patient's blood, e.g., that is not inherently necessary to withdraw as part of the procedure. Flow switch 110 is configured to reduce the incidental withdrawal of the patient's blood during a aspiration procedure by reducing a flow of fluid through a catheter lumen of catheter 108.

More specifically, flow switch 110 is configured to control the flow of fluid through the catheter lumen between an open, or “high flow,” state, and a compressed, or “low flow,” state, e.g., to control and/or restrict aspiration of a body fluid from a body of a patient. Medical aspiration system 100 defines a continuous fluid flow pathway from distal opening 114 to discharge reservoir 104, and flow switch 110 is configured to reduce a flow rate through the continuous fluid flow pathway by compressing tubing positioned between distal opening 114 to discharge reservoir 104. For example, flow switch 110 can be positioned to directly compress elongated body 112 of catheter 108 or a flexible tube positioned between a proximal end of catheter 108 and discharge reservoir 104. In general, flow switch 110 is configured to actuate between restricting the continuous fluid flow pathway to inhibit or disrupt a proximal flow of a body fluid, such as blood, and allowing the proximal flow of the body fluid through the continuous pathway unrestricted, thereby enabling efficient aspiration of thrombus material into distal catheter opening 114 and proximally through a catheter lumen defined by elongated body 112 of catheter 108.

In some examples, flow switch 110 includes an anvil and an actuator, with at least one of the actuator or the anvil being configured to move towards the other of the anvil or the actuator to compress a flexible tube positioned between the actuator and the anvil, the flexible tube defining at least a portion of the continuous fluid flow pathway through medical aspiration system 100. At least one of the anvil or the actuator includes a surface feature configured to cause a channel within a lumen of the flexible tube to remain open when the flexible tube is compressed between the anvil and the actuator, e.g., thereby allowing a low flow of the fluid when the flow switch is in the compressed, low flow configuration.

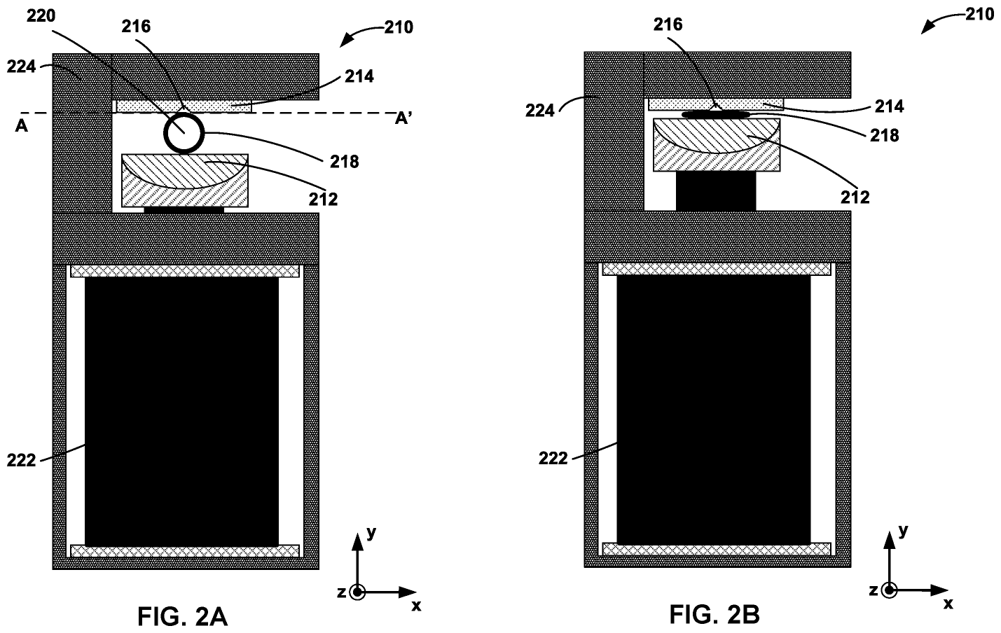

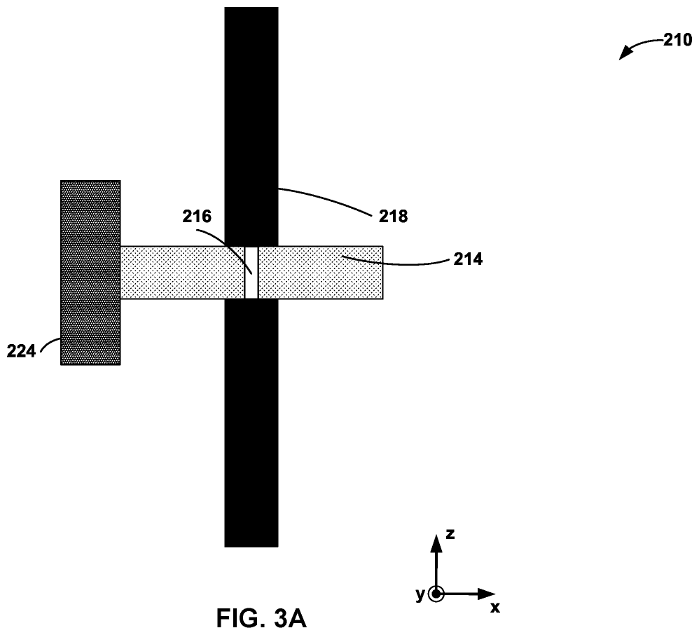

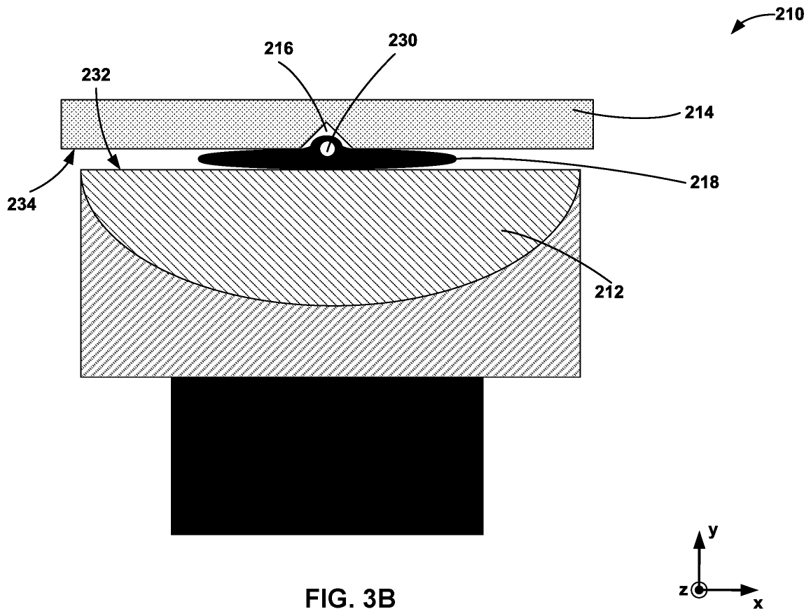

FIGS. 2A and 2B depict schematic side views of an example flow switch 210, which is an example of flow switch 110 of FIG. 1. In the example of FIG. 2A, flow switch 210 is depicted in a “high flow” or “open” configuration, in which an aspirated fluid or substance is freely able to flow proximally (e.g., in the z-direction from the perspective of FIG. 2A) through a continuous fluid pathway through tube lumen 220 of flexible tube 218. Flexible tube 218 is shown in cross-section in FIGS. 2A and 2B. In the example of FIG. 3B, flow switch 210 is depicted in a “low flow” or “compressed” configuration, in which the continuous pathway is obstructed to restrict fluid flow and/or limit the fluid flow rate through tube lumen 220, e.g., via compressing flexible tube 218 to reduce the cross-sectional area of lumen 220.

As shown in FIGS. 2A and 2B, flow switch 210 comprises actuator 212 and anvil 214 supported and held opposite each other by housing 224. Flexible tube 218 defines tube lumen 220 and is configured to be fluidically coupled to discharge reservoir 104 and a catheter lumen of catheter 108. In other examples, flexible tube 218 includes catheter 108. When flow switch 210 is in the open configuration, as shown in FIG. 2A, flexible tube 218 is configured to be positioned between actuator 212 and anvil 214 without being compressed.

In some examples, actuator 212 is configured to be actuated by solenoid 222, which may also be supported and held by housing 224. In some examples, solenoid 222 may be some other type of motivator, e.g., solenoid 222 may not be a solenoid, but rather a pneumatic actuator, a hydraulic actuator, a stepper actuator (e.g., driven by a stepper motor), a servo motor, or any suitable motivator configured to cause actuator 212 (and/or anvil 214) to move. In some examples, anvil 214 is configured to be actuated (e.g., by solenoid 222, a motor, or the like), and in some examples both actuator 212 and anvil 214 may be configured to be actuated and move towards or away from each other. Solenoid 222 may be configured to move and/or hold actuator 212 to be separated from anvil 214 (and/or move and/or hold anvil 214 to be separated from actuator 212) in the open configuration illustrated in FIG. 2A. Solenoid 222 may also be configured to move and/or hold actuator 212 towards anvil 214 (or anvil 214 towards actuator 212, or both anvil 214 and actuator 212 towards each other), e.g., in the compressed configuration (FIG. 2B). For example, solenoid 222 may be configured to press actuator 212 towards anvil 214 (or anvil 214 towards actuator 212, or both actuator 212 and anvil 214 towards each other) with at least a threshold amount of force. In some examples, solenoid 222 is configured to move actuator 212 (or anvil 214) to only two positions, e.g., a first position corresponding to the open configuration and a second position corresponding to the closed configuration in which actuator 212 and anvil 214 are closer to each other. In other examples, actuator 212 and/or anvil 214 is configured to be actuated and/or moved by a motorized valve, an electrostatic actuator, a pneumatic or hydraulic actuator, or any suitable actuator configured to actuate and/or move actuator 212 and/or anvil 214, e.g., additionally or alternatively to solenoid 222.

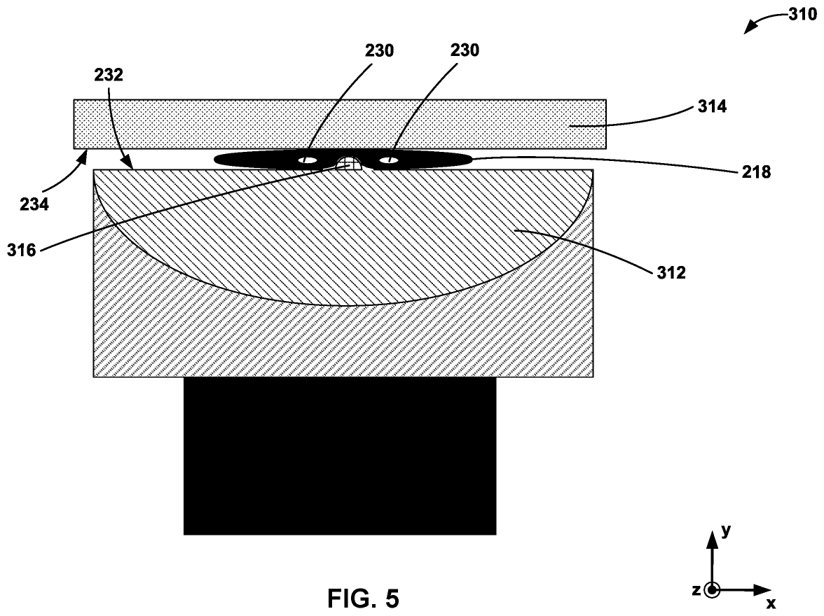

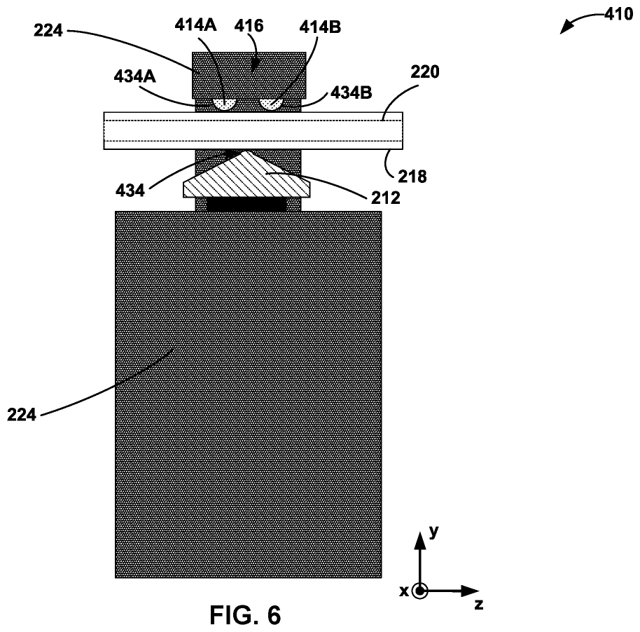

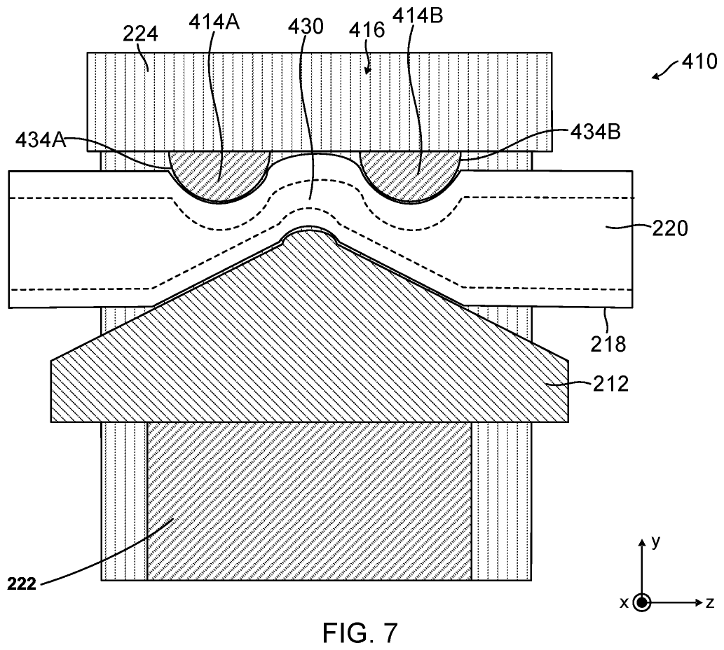

At least one of actuator 212 or anvil 214 includes a surface feature 216 configured to cause a channel within lumen 220 of flexible tube 218 to remain open when flexible tube 218 is compressed between actuator 212 and anvil 214, e.g., in the compressed configuration. FIGS. 2A-7 illustrate example surface features 216-416. In the example shown in FIGS. 2-4B, anvil 214 includes surface feature 216, which is a recess defined by a surface of anvil 214 facing actuator 212. FIG. 5 illustrates example surface feature 316 as a protrusion defined by a surface (or otherwise on a surface) of actuator 312 facing anvil 314, and FIGS. 6-7 illustrate surface feature 416 comprising a recess defined by anvil 414 extending in a direction along a longitudinal anvil axis or as a plurality of substantially curvilinear surfaces having longitudinal anvil axes and separated by gap distances along the longitudinal tube axis. In other examples, one or both of an actuator or an anvil includes a surface feature configured to allow a fluid to flow within a lumen of the flexible tube with a flow switch in a compressed and/or closed configuration, e.g., the surface feature may be any or all of a recess, a protrusion, a surface structure defining a surface profile, or the like.

FIG. 3A is a schematic cross-sectional top view of an example of flow switch 210 along the line A-A′ shown in FIG. 2A, and illustrates the relative directions of anvil 214, flexible tube 218, and surface feature 216. In the example shown, surface feature 216 extends in a direction along the longitudinal tube axis of flexible tube 218, e.g., along the z-axis as shown.

FIG. 3B is a schematic side view of actuator 212 and anvil 214 of flow switch 210 in the compressed or low flow configuration, and illustrates flexible tube 218 in cross-section. Other portions of the system, such as housing 224, are not shown in FIG. 3B, as well as FIGS. 5 and 6. In the example shown in FIG. 3B, surface feature 216 has a triangular cross-sectional shape in the x-y plane (e.g., a plane perpendicular to a longitudinal axis of flexible tube 218 and substantially within a plane including a longitudinal axis of anvil 214), although in other examples, surface feature 216 may have any other suitable cross-sectional shape, e.g., circular, elliptical, rectangular or square, or the like. In some examples, surface feature 216 is configured to have blunted, chamfered, or otherwise smoothed edges. For example, although surface feature 216 is illustrated in FIG. 3B as having sides terminating in sharp angles, the edges (e.g., at the base and apex of the triangular recess), the edges where surfaces of the cross-sectional shape of surface feature 216 meet may be curved, angled, or otherwise softened, and surface feature 216 may be configured to not have sharp edges.

As shown in FIG. 3B, in the compressed configuration, a portion flexible tube 218 aligned with surface feature 216 is subject to a reduced compressive force, e.g., by virtue of material of anvil 214 being removed at that portion. The portion of flexible tube 218 may move into (e.g., be compressed and or forced into) surface feature 216 and allow a portion of lumen 220 to remain open. For example, a portion of flexible tube 218 may expand into surface feature 216, allowing a portion of lumen 220 to remain open (e.g., via the elasticity of the flexible tube being allow to partially, or slightly, return to its original, uncompressed shape via surface feature 216) and not compressed, allowing at least a portion of lumen 220 to form a fluid channel 230. In other words, surface feature 216 is configured to cause channel 230 within lumen 220 of flexible tube 218 to remain open when flexible tube 218 is compressed by actuator 212 and anvil 214 in the compressed. In some examples, flow switch 210 is configured to have two configurations, the open configuration and the compressed configuration in which flow switch 210 allows some fluid flow but is not an intermediate configuration or state between “open” and “closed.” For example, flow switch 210 is configured such that the compressed configuration of flow switch 210 is effectively “fully closed” (thereby simplifying the complexity of the flow switch 210 relative to a flow switch configured to have three or more configurations or states) while still allowing a relatively low flow of fluid, e.g., to reduce clot formation within tubing or the catheter, and/or boiling and/or foaming within the discharge reservoir.

In some examples, surface feature 216 extends in a direction along a longitudinal tube axis of flexible tube 218. In the examples shown in FIGS. 2A-6, the longitudinal tube axis of flexible tube 218 extends along the z-axis direction, and surface feature 216 extends in generally the z-direction (e.g., parallel to the z-axis or within 5 or 10 degrees or less of the z-axis). This enables channel 230 to generally extend along the longitudinal tube axis of flexible tube 218 and allow fluid to flow through flow switch 210 in the compressed configuration via channel 230 of lumen 220 within flexible tube 218. Orthogonal x-y-z axes are shown in the figures for ease of description.

In some examples, actuator 212 and/or anvil 214 may comprise a substantially curved surface. For example, one or both of surfaces 232 and 234 may be substantially curved in at least one dimension, e.g., a two-dimensional (2D) curved surface such as a cylindrical surface, a three-dimensional (3D) and/or compound curved surface such as a spherical surface, a 2D or 3D curvilinear surface, or any suitable curve or shape. In the example shown in FIGS. 2A-7, at least a portion of actuator surface 232 is substantially curved along the z-axis direction and is substantially linear (e.g., straight) along the x-axis direction (e.g., a cylindrical surface having a cylindrical actuator axis substantially perpendicular to the longitudinal tube axis of flexible tube 218). In the examples shown, at least a portion of anvil surface 234 is substantially curved along the z-axis direction and is substantially linear (e.g., straight) along the x-axis direction (e.g., a cylindrical surface having a cylindrical actuator axis substantially perpendicular to the longitudinal tube axis of flexible tube 218). In some examples, substantially curved surfaces 232, 234 face each other and are configured to provide a contact area to compress flexible tube 218 substantially without sharp edges, corners, and/or cause localized stress areas on the material of flexible tube 218 in the compressed configuration.

In some examples, flow switch 210 is configured such that an apex of actuator surface 232 (e.g., in the y-axis direction) is positioned substantially opposite an apex of anvil surface 234 (e.g., in the y-axis direction). For example, the apices of surfaces 232 and 234 may be substantially aligned in the z-axis direction such that the apices of surfaces 232, 234 are the closest points of actuator 212 and anvil 214 along surfaces 232, 234 in the compressed configuration. For example, in some examples, flow switch 210 is configured to compress a portion of the length of flexible tube 218 in the z-axis direction in the compressed configuration without bending and/or moving the longitudinal axis of flexible tube 218 in the y-direction and or the x-direction. In other examples, flow switch 210 may be configured such that apices of surfaces 232, 234 are not necessarily aligned and are not necessarily the closest points along surfaces 232, 234 in the compressed configuration. For example, flow switch 210 may be configured to compress a portion of the length of flexible tube 218 in the z-direction in the compressed configuration while bending and/or moving the longitudinal axis of flexible tube 218 in the y-direction and or the x-direction.

Actuator 212 and anvil 214 comprise any material configured to compress flexible tube 218. For example, actuator 212 and anvil 214 may comprise a metal, a polymer, a rubber, wood, a ceramic, or any material capable of compressing flexible tube 218. Actuator 212 and anvil 214 may comprise the same material or may each comprise a material different from each other.

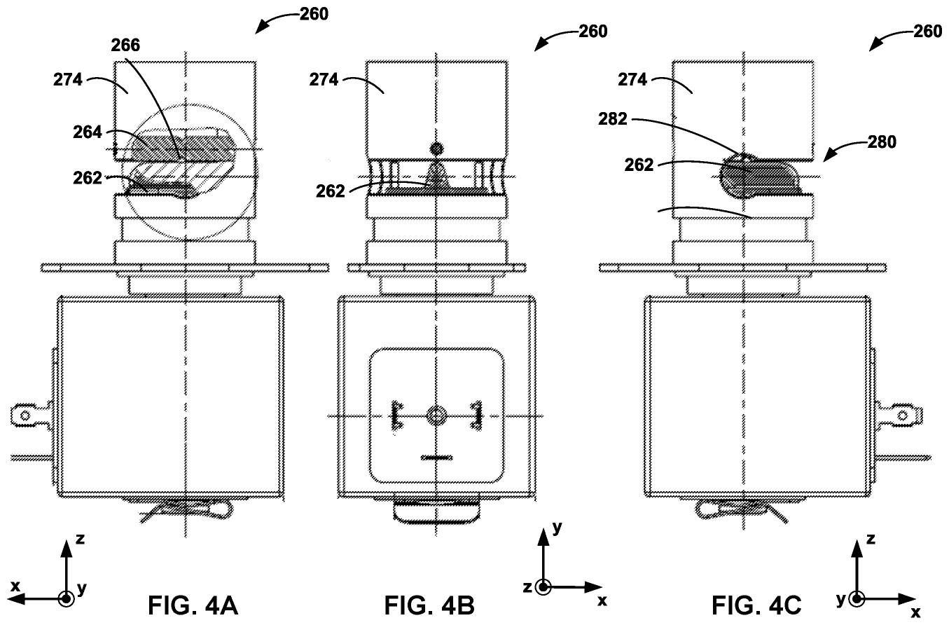

FIGS. 4A-4C are schematic views of an example flow switch 260. FIG. 4A is a schematic side view of flow switch 260, FIG. 4B is a schematic front view of flow switch 260, and FIG. 4C is a schematic side view of flow switch 260 from the side opposing the side illustrated in FIG. 4A. Flow switch 260 may be substantially similar to flow switch 210 described above, and illustrates slot 280 and tube channel 282 for inserting and holding a flexible tube within the housing.

In the example shown, flow switch 260 comprises actuator 262 and anvil 264, which may be substantially similar to actuator 212 and anvil 214 described above, and which are supported and held opposite each other by housing 274, which may be substantially similar to housing 274 described above. In the example shown, anvil 264 includes surface feature 266, which may be substantially similar to surface feature 216 described above. In the example shown, housing 274 includes slot 280, which is configured to receive a flexible tube from the front of housing 274 into tube channel 282, which is configured to hold the flexible tube within flow switch 260, e.g., centered with respect to actuator 262 and anvil 264.

In some examples, flexible tube 218 may be held centered in valve 260 by mechanical supports at either side of valve 260. For example, the mechanical supports my include a narrow opening (e.g., slot 280 to insert and/or receive flexible tube 218) that opens channel 282 which may have substantially the same diameter as the outer diameter of flexible tube 218 and which may be configured to locate, position, and/or hold flexible tube 218 in the valve.

FIG. 5 is a schematic side view of actuator 312 and anvil 314 of flow switch 310 in the low flow configuration and illustrates flexible tube 218 in cross-section. In the example shown, surface feature 316 has a circular cross-sectional shape in the x-y plane (e.g., a plane substantially perpendicular to a longitudinal axis of flexible tube 218 and substantially within a plane including a longitudinal axis of anvil 314), although in other examples, surface feature 316 may have any other suitable cross-sectional shape, e.g., triangular, elliptical, rectangular or square, or the like. In some examples, surface feature 316 is configured to have blunted, chamfered, or otherwise smoothed edges, similar to surface feature 216 describe above.

As shown in FIG. 5, in the compressed configuration, a portion flexible tube 218 aligned with surface feature 216 is subject to an increased compressive force, e.g., by virtue of surface feature 316 protruding into a portion flexible tube 218. The increased compressive force due to surface feature 316 may cause the material of flexible tube 218 to deform and stretch, which may cause one or more channels, e.g., channels 330A and/or 330B (collectively “channels 330”), to open or remain open within lumen 220. In other words, surface feature 316 is configured to cause one or more channels 330 within lumen 220 of flexible tube 218 to remain open when flexible tube 218 is compressed by actuator 312 and anvil 314 in the compressed configuration. In some examples, flow switch 310 is configured to have two configurations, the open configuration and the compressed configuration in which flow switch 310 allows some fluid flow but is not an intermediate configuration or state between “open” and “closed.” For example, flow switch 310 is configured such that the compressed configuration of flow switch 310 is effectively “fully closed” (thereby simplifying the complexity of the flow switch 310 relative to a flow switch configured to have three or more configurations or states) while still allowing a relatively low flow of fluid, e.g., to reduce clot formation within tubing or the catheter, and/or boiling and/or foaming within the discharge reservoir.

In some examples, surface feature 316 extends in a direction along a longitudinal tube axis of flexible tube 218 when flexible tube 218 is positioned between actuator 312 and anvil 314. In the examples shown in FIG. 5, the longitudinal tube axis of flexible tube 218 is along the z-axis direction, and surface feature 316 extends generally in the z-axis direction (e.g., parallel to the z-axis or within 5 or 10 degrees or less of the z-axis). This enables channel 230 to extend along the longitudinal tube axis of flexible tube 218 and allow fluid to flow through flow switch 310 in the compressed configuration via channel(s) 330 of lumen 220 within flexible tube 218.

In some examples, actuator 312 and/or anvil 314 may comprise substantially curved surfaces 232 and 234, respectively, as described above. Actuator 312 and anvil 314 may comprise any material configured to compress flexible tube 218. For example, actuator 312 and anvil 314 may comprise a metal, a polymer, a rubber, wood, a ceramic, or any material capable of compressing flexible tube 218. Actuator 312 and anvil 314 may comprise the same material or may each comprise a material different from each other.

FIGS. 6 and 7 depict side views of an example flow switch 410, which is an example of flow switch 110 of FIG. 1, and illustrates flexible tube 218 in cross-section. In the example of FIG. 6, flow switch 410 is depicted in a “high flow” or “open” configuration, in which an aspirated fluid or substance is freely able to flow proximally (e.g., in the z-direction) through tube lumen 220 of flexible tube 218. In the example of FIG. 7, flow switch 410 is depicted in a “low flow” or “compressed” configuration, in which the continuous pathway is obstructed to restrict fluid flow and/or limit the fluid flow rate through tube lumen 220, e.g., via both compressing flexible tube 218 to reduce the cross-sectional area of lumen 220 and “kinking” and/or causing flexible tube 218 to serpentine about anvil 414, actuator 412, and/or surface feature 416.

As shown in FIGS. 6 and 7, flow switch 410 comprises actuator 412 and anvil 414 supported and held opposite each other by housing 224. Housing 224 may be substantially similar as described above. Flexible tube 218 is configured to be positioned between actuator 412 and anvil 414 without being compressed in the open configuration. For example, actuator 412 may be actuated by solenoid 222, which may also be supported and held by housing 224. Solenoid 222 may be configured to move and/or hold actuator 412 to be separated from anvil 414 in the open configuration illustrated in FIG. 6. Solenoid 222 may also be configured to move and/or hold actuator 412 towards anvil 414, e.g., in the compressed configuration (FIG. 7). For example, solenoid 222 may be configured to press actuator 412 towards anvil 416 with at least a threshold amount of force. In some example, solenoid 222 may be configured to move actuator 412 to only two positions, e.g., a first position corresponding to the open configuration and a second position corresponding to the closed configuration in which actuator 412 is closer to anvil 414. In other examples, actuator 412 may be actuated and/or moved by a motorized valve, an electrostatic actuator, a pneumatic or hydraulic actuator, or any suitable actuator configured to actuate and/or move actuator 412, e.g., additionally or alternatively to solenoid 222.

At least one of actuator 412 or anvil 414 include a surface feature 416 configured to cause a channel within tube lumen 220 of flexible tube 218 to remain open when flexible tube 218 is compressed between actuator 212 and anvil 214, e.g., in the compressed configuration illustrated in FIG. 7. Surface feature 416 may comprise a surface structure, and surface profile, a surface pattern, or the like, of one or both of actuator 412 and anvil 414. In some examples, the surface structure, surface profile, surface pattern, or the like, may be along the longitudinal tube axis, e.g., along the z-direction.

In some examples, surface feature 416 is defined by a plurality of anvils having anvil axes substantially perpendicular to the tube axis (e.g., anvil axes in the x-axis direction as shown), e.g., which may form an anvil surface profile along the longitudinal tube axis (in the z-direction as shown). For example, surface feature 416 may comprise a recess defined by anvil 414 that extends in a direction along the longitudinal anvil axis (e.g., the x-axis direction as shown. In the examples shown, surface feature 416 is a channel in the surface of anvil 414, which defines or otherwise includes anvil structures 414A and 414B.

In other examples, surface feature 416 may be defined by a plurality of anvils, e.g., anvil structures 414A and 414B may be physically separate anvils 414A and 414B comprising a first substantially curvilinear surface 434A having a first longitudinal anvil axis that is perpendicular to a longitudinal tube axis of the flexible tube and a second substantially curvilinear surface 434B having a second longitudinal anvil axis that is perpendicular to the longitudinal tube axis, and first and second substantially curvilinear surfaces 434A and 434B are separated from each other by a gap distance along the tube axis, e.g., in the z-direction. Actuator 412 may include curvilinear surface 432, and as illustrated in FIG. 7, surface feature 416 may be configured to restrict the flow of a fluid through lumen 220 via compressing lumen 220 and causing lumen 220 to serpentine and/or curve (e.g., in the y-z plane as shown), while allowing at least a channel (e.g., channel 430) to remain open, when flow switch 410 is in the compressed or low flow configuration.

In the example shown, surface feature 416 includes two anvil structures (or anvils 434A and 434B. In other examples, surface feature 416 may include a plurality of structures, and/or a surface profile along the tube axis (e.g., in the z-direction) such as a square or rectangle wave, a triangle wave, a sinusoidal wave, a cylindrical wave, or any surface profile shape. In some examples, actuator 212 may additionally or alternatively comprise such a surface profile. For example, actuator 412 and anvil 414 may each comprise a plurality of anvil structures 434A, 434B (with the anvil structures on actuator 412 being 180 degrees rotated relative to anvil structures 414A and 414B shown, e.g., with an apex in the positive y-direction) which may be shifted or offset relative to each other, effectively forming anvil and actuator surface features that fit at least partially within the gaps between opposing actuator and anvil surface features and causing lumen 220 to have multiple curves along a greater length along the tube axis and to compress lumen 220 along a greater length along the tube axis. In some examples, a surface profile having a greater longitudinal length of compression of lumen 220 and multiple curves may have a greater repeatability and/or precision in achieving a desired fluid flow rate through flow switch 410 in the compressed configuration and may provide a greater longevity of flexible tube 218. For example, surface feature 416 may be configured to spread compression of flexible tube 218 out to a greater surface area of flexible tube 218 with less compression to achieve a similar flow restrictions through lumen 220, thereby causing reduced stress on the material of flexible tube 218. In other examples, flow switch 210 and/or 310 may be simpler, easier to fabricate, and lower cost, and configured such that flexible tube 218 is replaceable.

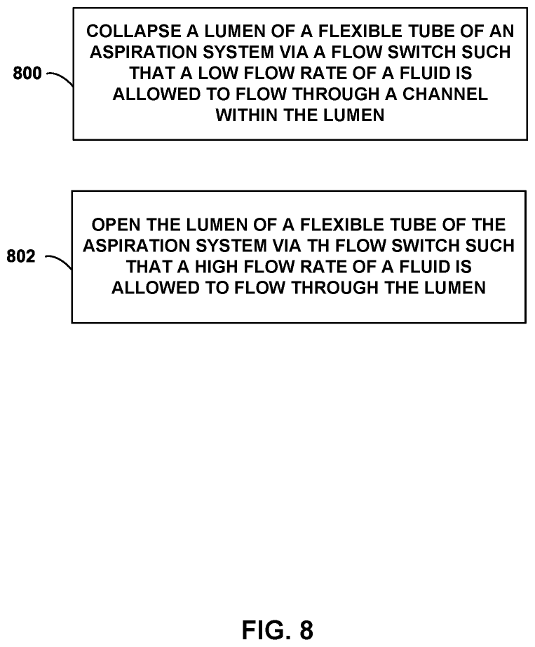

Flow switches as described herein may be formed using any suitable technique and can be used in any suitable medical procedure. FIG. 8 is a flow diagram of an example technique for using the aspiration systems and flow switches described herein. The technique of FIG. 8 is described with reference to the various aspects of medical aspiration system 100 of FIG. 1 and flow switch 210 of FIGS. 2A-4B for illustrative purposes, however, such descriptions are not intended to be limiting. The technique of FIG. 8 may be used with other aspiration systems and/or flow switches described herein, e.g., flow switches 310 and/or 410, or medical aspiration system 100 and/or flow switch 210 of FIG. 1 may be used using techniques other than those described with reference to FIG. 8.

In accordance with the technique shown in FIG. 8, a flow switch collapses a lumen of a flexible tube to a compressed configuration such that a flow rate of a fluid through a channel within the lumen is less than a flow rate of a fluid through the lumen in an open configuration (800). For example, a clinician fluidically may couple aspiration tubing, including flexible tube 218, to a suction source 102 and to the inner lumen of a catheter 108. The clinician may mechanically and fluidically connect vacuum tube 116 to a proximal opening of flow switch 210. The clinician may also mechanically and fluidically connect elongated body 112 of catheter 108 to distal opening of flow switch 210, so as to define a continuous fluid flow pathway through the inner lumen of catheter 108, the lumen 220 of flexible tube 218 of flow switch 210, and the inner lumen of vacuum tube 116.

Prior to or after coupling flexible tube 218 to catheter 108 and suction source 102, the clinician may introduce catheter 108 into vasculature of a patient and navigate catheter 108 to a target treatment site within a patient. In some examples, the clinician may navigate catheter 108 to the target site with the aid of a guidewire, guide catheter or another guide member.

After distal opening 114 of catheter 108 is positioned as desired proximate a thrombus in the vasculature (e.g., as indicated via fluoroscopic imagery), control circuitry 120, alone or based on input from a user received via a user input device, controls suction source 102 to generate a suction force within the inner lumen of catheter 108.

In some instances, distal opening 114 of catheter 108 may initially be positioned near thrombus material, but may not be positioned against (e.g., occluded by) the thrombus material. In such instances, the suction force from suction source 102 may begin to aspirate a patient fluid, such as blood, into catheter 108, generating a proximal fluid flow through flow switch 210, e.g., via lumen 229 of flexible tube 218. Automatically (e.g., based on a sensed condition) or in response to user input, control circuitry 120 controls solenoid 222 to cause actuator 212 to move to the compressed configuration, compressing flexible tube 218 and thereby restricting fluid flow through flow switch 210, but allowing at least some fluid to flow through lumen 220 via channel 230 which is caused to remain open by surface feature 216.

The clinician may continue to manipulate distal opening 114 of catheter 108 until distal opening 114 is positioned against, and occluded by, thrombus material. At such time, the fluid flow into catheter 108 will be disrupted by the thrombus material at distal opening 114, and control circuitry 120 may cause solenoid 222 to cause actuator 212 to move to open lumen 220 of flexible tube 218 such that a high flow rate of a fluid is allowed to flow through lumen 220 (802). With flow switch 210 in the open configuration, the clinician may proceed to aspirate the thrombus and remove catheter 108 from the vasculature of the patient once the procedure is complete.

The techniques described in this disclosure, including those attributed to control circuitry 120, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example. In addition, analog circuits, components and circuit elements may be employed to construct one, some or all of the control circuitry 120, instead of or in addition to the partially or wholly digital hardware and/or software described herein. Accordingly, analog or digital hardware may be employed, or a combination of the two. Whether implemented in digital or analog form, or in a combination of the two, control circuitry 120 can comprise a timing circuit.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

The following clauses provide some examples of the disclosure. The examples described herein may be combined in any permutation or combination.

Example 1: A medical system for aspirating material from a patient, the device including: a flow switch including: an anvil; an actuator; and a surface feature on at least one of the anvil and actuator, wherein the flow switch is configured to: move the actuator away from the anvil create a flow path for the aspiration of the material; and move the actuator toward the anvil to reduce the flow path by creating at least one channel defined by the surface feature.

Example 2: The medical system of example 1, further comprising a flexible tube coupled to an elongated body of a catheter that is configured to aspirate the material from the patient and wherein the material is thrombus.

Example 3: The medical system of example 2, wherein the actuator and the anvil are configured to compress the flexible tube from an open configuration to a compressed configuration, wherein when the flexible tube is in the open configuration and is fluidically coupled to the elongated body of the catheter, a suction source that is fluidically coupled to the flexible tube is configured to cause a first flow rate through the lumen of the flexible tube, and wherein the surface feature is configured to cause the channel within the lumen of the flexible tube to remain open when the flexible tube is compressed such that the channel allows a second flow rate through the lumen that is greater than zero and less than or equal to 5% of the first flow rate.

Example 4: The medical system of example 2 or example 3, wherein a surface of the at least one of the actuator or the anvil defines the surface feature, wherein the surface feature comprises at least one of a protrusion extending outwardly from the surface or a recess extending inwardly from the surface.

Example 5: The medical system of example 4, wherein the protrusion or the recess extends in a direction along a longitudinal tube axis of the flexible tube to cause the channel to extend along the longitudinal tube axis.

Example 6: The medical device of any one of examples 2 through 5, wherein the anvil comprises a substantially curvilinear surface having a longitudinal anvil axis that is perpendicular to a longitudinal tube axis of the flexible tube to cause the channel to extend along the longitudinal tube axis.

Example 7: The medical system of example 6, wherein the surface feature comprises a recess defined by the anvil that extends in a direction along the longitudinal anvil axis.

Example 8: The medical system of any one of examples 2 through 7, wherein the anvil comprises the surface feature, wherein the surface feature comprises a first substantially curvilinear surface having a first longitudinal anvil axis that is perpendicular to a longitudinal tube axis of the flexible tube and a second substantially curvilinear surface having a second longitudinal anvil axis that is perpendicular to the longitudinal tube axis, wherein the first and second substantially curvilinear surfaces are separated from each other by a gap distance along the tube axis.

Example 9: The medical system of any one of examples 1 through 8 further including: a suction source; and a catheter in fluidic connection to the suction source and designed for use within a peripheral vascular system of the patient.

Example 10: A medical aspiration system including: a suction source; an elongated body defining a lumen fluidically coupled to the suction source; and a pinch valve configured to actuate between a high flow configuration and a low flow configuration, wherein in the low flow configuration the pinch valve is configured to compress the elongated body while still enabling fluid flow through the lumen.

Example 11: The medical aspiration system of example 10, wherein the pinch valve is configured to allow a first flow rate through the lumen in the high flow configuration and to allow a second flow rate through the lumen in the low flow configuration that is less than or equal to 5% of the first flow rate.

Example 12: The medical aspiration system of example 10 or example 11, wherein the pinch valve comprises: an anvil; and an actuator, wherein the elongated body is positioned between the actuator and the anvil, wherein at least one of the actuator or the anvil are configured to move towards the other of the anvil or the actuator to compress the elongated body, wherein at least one of the anvil or the actuator comprises a surface feature configured to cause a channel within the lumen to remain open when the elongated body is compressed between the anvil and the actuator.

Example 13: The medical aspiration system of example 12, wherein a surface of the at least one of the actuator or the anvil defines the surface feature comprising at least one of a protrusion extending outwardly from the surface or a recess extending inwardly from the surface.

Example 14: The medical aspiration system of example 13, wherein the protrusion or the recess extends in direction along a longitudinal body axis of the elongated body to cause the channel to extend along the longitudinal body axis.

Example 15: The medical aspiration system of any one of examples 12 through 14, wherein the anvil comprises a substantially curvilinear surface having a longitudinal anvil axis that is perpendicular to a longitudinal body axis of the elongated body to cause the channel to extend along the longitudinal body axis.

Example 16: The medical aspiration system of example 15, wherein the surface feature comprises a recess defined by the anvil that extends in a direction along the longitudinal anvil axis.

Example 17: The medical aspiration system of any one of examples 12 through 16, wherein the anvil comprises the surface feature, wherein the surface feature comprises a first substantially curvilinear surface having a first longitudinal anvil axis that is perpendicular to a longitudinal body axis of the elongated body and a second substantially curvilinear surface having a second longitudinal anvil axis that is perpendicular to the longitudinal body axis, wherein the first and second substantially curvilinear surfaces are separated from each other by a gap distance along the longitudinal body axis.

Example 18: The medical aspiration system of any one of examples 12 through 17, wherein the pinch valve comprises a solenoid configured to actuate the actuator to only the high flow configuration or the low flow configuration.

Example 19: A method for aspirating material from a patient using a flow switch, an anvil, an actuator, and a surface feature on at least one of the anvil and actuator, the method including: opening the flow switch by moving the actuator away from the anvil to create a flow path for the aspiration of the material; and closing the flow switch by move the actuator toward the anvil to reduce the flow path by creating at least one channel defined by the surface feature.

Example 20: The method of example 19, wherein closing the flow switch includes creating the at least one channel within a lumen of a flexible tube, the lumen defined by at least one of a protrusion extending outwardly from the surface and a recess extending inwardly from the surface.

Example 21: The method of example 20, wherein opening and closing the flow switch includes using a solenoid to move the actuator between only the compressed configuration or the open configuration.

Various aspects of the disclosure have been described. These and other aspects are within the scope of the following claims.

What is claimed is:

1. A medical system for aspirating material from a patient, the system comprising:
a flow switch comprising:
an anvil;
an actuator; and
a surface feature on at least one of the anvil and actuator, wherein the flow switch is configured to:
move the actuator away from the anvil create a flow path for the aspiration of the material; and
move the actuator toward the anvil to reduce the flow path by creating at least one channel defined by the surface feature.

2. The medical system of claim 1, further comprising a flexible tube coupled to an elongated body of a catheter that is configured to aspirate the material from the patient and wherein the material is thrombus.

3. The medical system of claim 2, wherein the actuator and the anvil are configured to compress the flexible tube from an open configuration to a compressed configuration, wherein when the flexible tube is in the open configuration and is fluidically coupled to the elongated body of the catheter, a suction source that is fluidically coupled to the flexible tube is configured to cause a first flow rate through the lumen of the flexible tube, and wherein the surface feature is configured to cause the channel within the lumen of the flexible tube to remain open when the flexible tube is compressed such that the channel allows a second flow rate through the lumen that is greater than zero and less than or equal to 5% of the first flow rate.

4. The medical system of claim 2, wherein a surface of the at least one of the actuator or the anvil defines the surface feature, wherein the surface feature comprises at least one of a protrusion extending outwardly from the surface or a recess extending inwardly from the surface.

5. The medical system of claim 4, wherein the protrusion or the recess extends in a direction along a longitudinal tube axis of the flexible tube to cause the channel to extend along the longitudinal tube axis.

6. The medical system of claim 2, wherein the anvil comprises a substantially curvilinear surface having a longitudinal anvil axis that is perpendicular to a longitudinal tube axis of the flexible tube to cause the channel to extend along the longitudinal tube axis.

7. The medical system of claim 6, wherein the surface feature comprises a recess defined by the anvil that extends in a direction along the longitudinal anvil axis.

8. The medical system of claim 2, wherein the anvil comprises the surface feature, wherein the surface feature comprises a first substantially curvilinear surface having a first longitudinal anvil axis that is perpendicular to a longitudinal tube axis of the flexible tube and a second substantially curvilinear surface having a second longitudinal anvil axis that is perpendicular to the longitudinal tube axis, wherein the first and second substantially curvilinear surfaces are separated from each other by a gap distance along the tube axis.

9. The medical system of claim 1 further comprising:
a suction source; and
a catheter in fluidic connection to the suction source and designed for use within a peripheral vascular system of the patient.

10. A medical aspiration system comprising:
a suction source;
an elongated body defining a lumen fluidically coupled to the suction source; and
a pinch valve configured to actuate between a high flow configuration and a low flow configuration, wherein in the low flow configuration the pinch valve is configured to compress the elongated body while still enabling fluid flow through the lumen.

11. The medical aspiration system of claim 10, wherein the pinch valve is configured to allow a first flow rate through the lumen in the high flow configuration and to allow a second flow rate through the lumen in the low flow configuration that is less than or equal to 5% of the first flow rate.

12. The medical aspiration system of claim 10, wherein the pinch valve comprises:
an anvil; and
an actuator,
wherein the elongated body is positioned between the actuator and the anvil, wherein at least one of the actuator or the anvil are configured to move towards the other of the anvil or the actuator to compress the elongated body,
wherein at least one of the anvil or the actuator comprises a surface feature configured to cause a channel within the lumen to remain open when the elongated body is compressed between the anvil and the actuator.

13. The medical aspiration system of claim 12, wherein a surface of the at least one of the actuator or the anvil defines the surface feature comprising at least one of a protrusion extending outwardly from the surface or a recess extending inwardly from the surface.

14. The medical aspiration system of claim 13, wherein the protrusion or the recess extends in direction along a longitudinal body axis of the elongated body to cause the channel to extend along the longitudinal body axis.

15. The medical aspiration system of claim 12, wherein the anvil comprises a substantially curvilinear surface having a longitudinal anvil axis that is perpendicular to a longitudinal body axis of the elongated body to cause the channel to extend along the longitudinal body axis.

16. The medical aspiration system of claim 15, wherein the surface feature comprises a recess defined by the anvil that extends in a direction along the longitudinal anvil axis.

17. The medical aspiration system of claim 12, wherein the anvil comprises the surface feature, wherein the surface feature comprises a first substantially curvilinear surface having a first longitudinal anvil axis that is perpendicular to a longitudinal body axis of the elongated body and a second substantially curvilinear surface having a second longitudinal anvil axis that is perpendicular to the longitudinal body axis, wherein the first and second substantially curvilinear surfaces are separated from each other by a gap distance along the longitudinal body axis.

18. The medical aspiration system of claim 12, wherein the pinch valve comprises a solenoid configured to actuate the actuator to only the high flow configuration or the low flow configuration.

19. A method for aspirating material from a patient using a flow switch, an anvil, an actuator, and a surface feature on at least one of the anvil and actuator, the method comprising:
opening the flow switch by moving the actuator away from the anvil to create a flow path for the aspiration of the material; and
closing the flow switch by move the actuator toward the anvil to reduce the flow path by creating at least one channel defined by the surface feature.

20. The method of claim 19, wherein closing the flow switch includes creating the at least one channel within a lumen of a flexible tube, the lumen defined by at least one of a protrusion extending outwardly from the surface and a recess extending inwardly from the surface.

21. The method of claim 20, wherein opening and closing the flow switch includes using a solenoid to move the actuator between only the compressed configuration or the open configuration.
